(19) United States
(12) Patent Application Publication
(10) Pub. No.: US 2023/0086833 A1
(43) Pub. Date: Mar. 23, 2023

(54) DEFIBROTIDE TREATMENT FOR THE PREVENTION OF ORGAN REJECTION AND INJURY

(71) Applicant: Jazz Pharmaceuticals Ireland Limited, Dublin (IE)

(72) Inventors: Polly Rae PINE, Menlo Park, CA (US); Ranjesh HANVESAKUL, Solihull (GB)

(21) Appl. No.: 17/918,692

(22) PCT Filed: Apr. 16, 2021
(86) PCT No.: PCT/US2021/027804

§ 371 Date: Oct. 13, 2022

Related U.S. Application Data
(60) Provisional application No. 63/011,645, filed on Apr. 17, 2020.

(51) Int. Cl. A61K 31/711; A61P 9/10; A61K 9/00

(52) U.S. Cl. CPC A61K 31/711; A61P 9/10; A61K 9/0019

(57) ABSTRACT

The present disclosure provides methods of preventing, lessening the effects, or treating organ injury and/or organ rejection optionally associated with organ transplant comprising administering defibrotide. The defibrotide can be administered to the donor organ via the donor organ's artery, to the donor organ via a preservation bath, or to the patient.

### CROSS-REFERENCE TO RELATED APPLICATIONS

This application is a National Phase of International Application No. PCT/US2021/027804 filed Apr. 16, 2021, which claims the benefit to U.S. Provisional Application No. 63/011,645 filed Apr. 17, 2020. The contents of each of the aforementioned patent applications are hereby incorporated by reference in their entireties.

### FIELD

The present disclosure is directed to methods of administering defibrotide to prevent and/or treat organ rejection and injury, including ischemia reperfusion injury (IRI).

### BACKGROUND

There have been significant advances in organ transplantation over the previous decades, with progressive improvements made in the surgical technique, patient care, and most importantly, in the immunosuppression regimens provided to patients following transplantation leading to marked reductions in steroid-resistant graft rejection (from nearly 80% in the 1970s to well below 10% in 2011) and improvements in graft survival (3-year rates below 10% in the 1970s to over 80% in 2011) particularly among recipients of living kidney donor donation (data from Scientific Registry of Transplant Recipients). In the US, all-cause graft failure decreased from 7.5% in 2005 to 4.8% in 2015, and 10-year all cause graft failure decreased from 57.2% in 2006 to 51.6% in 2016. Still, the majority of recipients of organ transplants receive organs from deceased donors, which are associated with an increased risk of organ rejection and injury and an incidence of delayed graft function (DGF) ranging between 25-50%.

In patients with DGF that have received a kidney transplant, restriction of blood supply to the organ followed by restoration of blood flow and re-oxygenation leads to ischemia reperfusion injury (IRI), with tissue damage mediated by an inflammatory cascade initiated in response to the formation of reactive oxygen species (ROS), a decrease in glycolysis, a reduction in pH, and increase in ATP, and activating endothelial tissues. IRI, a type of acute kidney injury (AM), begins during initial donor management, progresses during organ procurement, and is exacerbated during transport, storage, implantation and reperfusion.

Brain death and agonal events (pre-death events in a state of hypoxia and other stress also lead to organ rejection and injury. For example, both warm ischemia and cold ischemia exacerbate organ injury, oftentimes contributing to delayed graft function (DGF). Warm ischemia is the storage of an organ at body temperature after its blood supply has been cut off, and cold ischemia is the time between the chilling of a tissue, organ, or body part after its blood supply has been reduced or cut off and the time it is warmed by having its blood supply restored. Damage to the donor organ occurs upon warming of the organ during anastomosis. The interplay of ischemic and non-ischemic injuries and subsequent innate and adaptive immune responses lead to the complex pathophysiologic mechanisms of organ injury in the allograft.

As a result of organ injury, the transplanted organ may not initially function as expected. Early allograft dysfunction may manifest variously, depending on a number of donor and recipient factors, along a continuum, from primary nonfunction (PNF) to slow graft function (SGF) and DGF.

Based on various donor and recipient risk factors, the risk of organ rejection and/or organ injury increases with the rate of delayed graft function being highest in the patients who receive donated organs from donors after cardiac death, compared with donors after brain death.

At the time of this disclosure, there are currently no FDA-approved treatments available to prevent or manage organ injury. Thus, there is a need for the development of new strategies to prevent and manage organ injury and/or organ failure.

### BRIEF DESCRIPTION OF THE DRAWINGS

FIG. 1 shows a dosing schedule for an in vivo study of defibrotide in dogs.

FIG. 2 shows a dose-dependent effect of defibrotide on hemorrhage after IRI in an in vivo study in dogs. Saline was used as a control.

FIG. 3 shows an effect of defibrotide administration on serum creatinine in dogs.

FIG. 4 shows defibrotide dosing arms for a Phase 1b study evaluating the safety and efficacy of defibrotide for the treatment and prevention of IRI in kidney transplant patients.

### SUMMARY OF THE INVENTION

In some aspects, the present disclosure provides methods of preventing, lessening the effects, or treating organ injury and/or organ rejection in a patient comprising administering a therapeutically effective amount of defibrotide. In some aspects, the present disclosure provides a method of preventing and/or lessening the effects of and/or treating organ injury and/or organ rejection in a patient comprising administering a therapeutically effective amount of defibrotide.

In some embodiments, the patient is receiving or about to receive or has received transplant of a donor organ or donor tissue.

In some embodiments, the donor organ is selected from the group consisting of a heart, heart valve, stomach, testis, liver, lungs, kidneys, pancreas, uterus, intestine, bladder, composite tissue, xenograft, and thymus, and/or wherein the donor tissue is selected from the group consisting of bones, bone marrow, tendons, corneae, skin, valves, nerves, and veins.

In some embodiments, the defibrotide is administered to the patient. In some embodiments, the defibrotide is administered to the donor organ intravenously via an artery. In some embodiments, the defibrotide is administered to a preservation bath that is used to store the organ for transfer.

In some embodiments, the defibrotide is administered before the development of organ injury and/or organ rejection. In some embodiments, the defibrotide is administered after the development of organ injury and/or organ rejection.

In some embodiments, the defibrotide is administered to the patient once per day. In some embodiments, the defibrotide is administered to the patient in multiple doses per day. In some embodiments, the defibrotide is administered in two to ten doses per day. In some embodiments, the defibrotide is administered to the patient four times per day.

In some embodiments, an initial dose of defibrotide is administered to the patient about 6 hours after transplant of a donor organ.

In some embodiments, the defibrotide is administered at a dose between 1 mg/kg and 10 mg/kg. In some embodiments, the defibrotide is administered at a dose of 2.5 mg/kg. In some embodiments, the defibrotide is administered at a dose of 6.25 mg/kg.

In some embodiments, the defibrotide is administered intravenously. In some embodiments, the defibrotide is administered subcutaneously. In some embodiments, the defibrotide is administered intravenously, every six hours at a dose of 2.5 mg/kg. In some embodiments, the defibrotide is administered intravenously, every six hours at a dose of 6.25 mg/kg. In some embodiments, the defibrotide is administered at a dose of 2.5 mg/kg. In some embodiments, the defibrotide is administered at a dose of 6.25 mg/kg.

In some embodiments, the defibrotide is a high concentration defibrotide formulation. In some embodiments, the high concentration defibrotide formulation is formulated for subcutaneous delivery or intravenous delivery. In some embodiments, the high concentration defibrotide formulation comprises about 160-200 mg/mL of defibrotide and about 20 mM glycylglycine, and is formulated for subcutaneous delivery to a patient. In some embodiments, the high concentration defibrotide formulation further comprises between about 10 mM to about 34 mM sodium citrate. In some embodiments, the high concentration defibrotide formulation comprises about 160-200 mg/mL of defibrotide, about 20 mM glycylglycine, and about 10-25 mM sodium citrate, wherein the formulation is formulated for subcutaneous or intravenous delivery to a patient.

In some embodiments, the organ injury is selected from the group consisting of interstitial fibrosis, tubular atrophy, glomerulosclerosis, acute kidney injury, diffuse alveolar damage, nonalcoholic steatohepatitis, fibrosis, cardiac allograph vasculopathy, and ischemia reperfusion injury (IRI).

### DETAILED DISCLOSURE

### Definitions

The term defibrotide identifies a polydeoxyribonucleotide that is obtained by extraction from animal and/or vegetable tissues but which may also be produced synthetically; the polydeoxyribonucleotide is normally used in the form of an alkali-metal salt, generally a sodium salt, and generally has a molecular weight of 13 to 30 kDa (CAS Registry Number: 83712-60-1). Preferably, defibrotide is obtained according to U.S. Pat. No. 4,985,552 and 5,223,609 and/or presents the physical/chemical characteristics described in the same U.S. Pat. Nos. 4,985,552 and 5,223,609, herein incorporated by reference. More in particular, defibrotide is a mixture of polydeoxyribonucleotides having formula of random sequence: P1-5, (dAP)12-24, (dGP)10-20, (dPp)13-26, (dCP)10-20, where: P=phosphoric radical; dAp=deoxyadenylic monomer; dGp=deoxyguanylic monomer; dTp=deoxythymidinic monomer; dCp=deoxycytidynic monomer; and/or shows the following chemical/physical characteristics: electrophoresis=homogeneous anodic mobility, and/or extinction coefficient, E₁ cm^{1%} at 260±1 nm nm=220±10, and/or E₂₃₀/E₂₆₀=0.45±0.04, and/or coefficient of molar extinction (referred to phosphorous) ε(P)=7.750±500, and/or rotatory power [α]_{D}²⁰°=53°±6; and/or reversible hyperchromicity, indicated as % in native DNA and/or h=15±5. In some embodiments, the defibrotide is Defitelio®.

The term “subject” is used interchangeably herein with “patient” to refer to an individual to be treated. The subject is a mammal (e.g., human, non-human primate, rat, mouse, cow, horse, pig, sheep, goat, dog, cat, etc.). The subject can be a clinical patient, a clinical trial volunteer, an experimental animal, etc. The subject can be suspected of having or at risk for having a condition or be diagnosed with a condition, such as organ injury and/or organ rejection. The subject can also be suspected of having or at risk for having organ injury and/or organ rejection. According to one embodiment, the subject to be treated according to this invention is a human. Subjects to be treated by the methods of the disclosed embodiments include both human subjects and animal subjects (e.g., dog, cat, monkey, chimpanzee, and/or the like) for veterinary purposes. The subjects may be male or female and may be any suitable age, e.g., neonatal, infant, juvenile, adolescent, adult, or geriatric. In some embodiments, the subjects are preferably mammalian.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

A “therapeutically effective amount”, as used herein refers to an amount that is sufficient to achieve a desired therapeutic effect. For example, a therapeutically effective amount can refer to an amount that is sufficient to improve at least one sign or symptom of organ injury and/or organ rejection.

The terms “a” and “an,” when used to modify the ingredient of a composition, such as, active agent, buffering agent, and osmolyte, do not denote a limitation of quantity, but rather denote the presence of at least one of the referenced item. The term “or” or “and/or” is used as a function word to indicate that two words or expressions are to be taken together or individually. The terms “comprising,” “having,” “including,” and “containing” are to be construed as open-ended terms (i.e., meaning “including, but not limited to”). The endpoints of all ranges directed to the same component or property are inclusive and independently combinable.

Throughout the present specification, the terms “about” and/or “approximately” may be used in conjunction with numerical values and/or ranges. The term “about” is understood to mean those values near to a recited value. For example, “about 1200 [units]” may mean within ±10% of 1200, within ±10%, ±9%, ±8%, ±7%, ±7%, ±5%, ±4%, ±3%, ±2%, ±1%, less than ±1%, or any other value or range of values therein. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 70-70, etc.). Furthermore, all values within a given range may be an endpoint for the range encompassed thereby (e.g., the range 50-80 includes the ranges with endpoints such as 55-80, 50-75, etc.).

The term, “excipient,” as used herein, refers to any substance that may be formulated with defibrotide and may be included for the purpose of enhancement of the defibrotide in the final dosage form, such as facilitating its bioavailability, reducing viscosity and/or osmolality, enhancing solubility of the composition or to enhance long-term stability. Excipients can also be useful in the manufacturing process, to aid in the handling of the active substance. The selection of appropriate excipients also depends upon the route of administration and the dosage form, as well as the active ingredient and other factors. Accordingly, defibrotide may be combined with any excipient(s) known in the art that allows tailoring its performance during manufacturing or administration as well as its in vitro and in vivo performance. Many of these excipients may be utilized to tailor the pharmacokinetic profiles of defibrotide formulations.

The term, “formulation,” as used herein, refers to compositions for therapeutic use, including, for example, a stable and pharmaceutically acceptable preparation of a pharmaceutical composition or formulation disclosed herein.

The term, “high concentration formulation” or “high concentration liquid formulation” or “HCLF” as used herein, refers to those formulations where the concentration of the nucleic acid is about 80 mg/mL or higher; or about 85 mg/mL or higher. In some aspects, the defibrotide is a high concentration, low viscosity defibrotide formulation. In some embodiments, the high concentration, low viscosity defibrotide formulation is one disclosed in U.S. application Ser. No. 16/105,319 filed Aug. 3, 2018 the contents of which are incorporated herein for all purposes.

The term, “high concentration defibrotide formulations” as used herein, refers to those formulations where the defibrotide concentration is about 80 mg/mL or higher, or about 85 mg/mL or higher.

The term, “pharmacokinetic” or “PK” as used herein, refers to in vivo movement of an individual agent in the body, including the plasma concentration time profiles and kinetic parameters like the maximum concentration (Cmax), area under the curve (AUC), and time to maximum concentration of said agent (Tmax).

The phrase “pharmaceutically acceptable” or “acceptable”, as used in connection with compositions of the disclosure, refers to molecular entities and other ingredients of such compositions that are physiologically tolerable and do not typically produce untoward reactions when administered to an animal and/or human. Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

As used herein, the term “parenteral” refers to any non-oral means of administration. It includes intravenous (i.v. or IV) infusion, IV bolus injection, subcutaneous (s.c. or SC) and intramuscular (i.m. or IM) injection. In some embodiments, defibrotide is administered intraveneously. In some embodiments, defibrotide is administered subcutaneously. Defibrotide which may be administered subcutaneously may require less frequent dosing than defibrotide products currently on the market are provided. In some embodiments, a device used to administer defibrotide is one disclosed in U.S. Application No. 62/776,500 filed Dec. 7, 2018 the contents of which are incorporated herein for all purposes. In some embodiments, a device used to administer defibrotide is one disclosed in U.S. Application No. 62/802,099 filed Feb. 6, 2019 the contents of which are incorporated herein for all purposes.

### Defibrotide and Its Mechanisms of Action

Defibrotide (CAS number 83712-60-1) is a substance derived from materials of natural origin. Defibrotide, a nucleic acid salt, is a highly complex mixture of random sequences, predominantly single-stranded polydeoxyribonucleotides (predominantly single stranded and approximately 10% double stranded) derived from animal mucosal DNA Defibrotide has pleotropic biologic effect leading to the stabilization of endothelial cells, and in particular, defibrotide has protective effects on vascular endothelial cells, particularly those of small vessels and has antithrombotic, anti-inflammatory and antiischemic properties.

Defibrotide has a diverse size range and is known to have a mean molecular weight (MW) between 13 and 20 kDa. Defibrotide can be obtained according to U.S. Pat. No. 4,985,552 and 5,223,609 and/or presents the physical/chemical characteristics described in the same U.S. Pat. No. 4,985,552 and 5,223,609, each of which is incorporated herein by reference. Synthetic defibrotide, presented as phosphodiester oligonucleotides that mimic the therapeutic action of defibrotide are described in US20110092576 which is incorporated herein by reference in its entirety.

Defibrotide has numerous therapeutic applications, including use as an anti-thrombotic agent (U.S. Pat. No. 3,829,567), treatment of peripheral arteriopathies (U.S. Pat. 5,081,109), treatment of acute renal insufficiency (U.S. Pat. No. 4,694,134), treatment of acute myocardial ischaemia (U.S. Pat. No. 4,693,995), topical treatments (U.S. Pat. No. 5,116,617) among other uses described in U.S. Pat. Nos. 3,770,720, 3,899,481, 4,938,873, 4,985,552, 5,223,609, 5,646,127, 5,646,268, and 6,046,172; all of the preceding patents are incorporated herein by reference in their entireties. More recently, defibrotide has been used for the treatment and prevention of sinusoidal obstruction syndrome/veno-occlusive disease (EU clinical trial EudraCT:2004-000592-33, US clinical trial 2005-01 (ClinicalTrials.gov identifier: NCT00358501). Defibrotide is currently sold under the name Defitelio® as a single vial for injection (commercially available from Gentium S.r.L., Villa Guardia, Italy; see package insert available at dailymed.nlm.nih.gov/dailymed/search.cfm?labeltype=all&query=defibrotide). Defitelio® is prepared as an intravenous infusion by a dilution in 5% Dextrose Injection, USP or 0.9% Sodium Chloride Injection, USP. Intravenous preparation is used within 4 hours if stored at room temperature or within 24 hours if stored under refrigeration. It is administered for a total of 8 hours over 4 intravenous infusions.

In some embodiments, defibrotide is administered as a “high concentration formulation” (HCLF). In some embodiments the concentration of the nucleic acid in the HCLF is about 80 mg/mL or higher; or about 85 mg/mL or higher. In some aspects, the defibrotide is a high concentration, low viscosity defibrotide formulation. In some embodiments, the high concentration, low viscosity defibrotide formulation is one disclosed in U.S. application Ser. No. 16/105,319 filed Aug. 3, 2018 the contents of which are incorporated herein for all purposes.

### Defibrotide Administration

In some embodiments, defibrotide may be administered to a patient undergoing, or about to undergo, an organ transplant. In some embodiments, defibrotide is administered to a patient that has received an organ transplant. In some embodiments, defibrotide is administered to prevent, ameliorate, delay, or treat organ injury or organ rejection in a patient undergoing, about to undergo, or having received an organ transplant.

In some embodiments, defibrotide is administered to a patient who is about to donate an organ. In some embodiments, defibrotide is administered before the patient donates an organ. In some embodiments, defibrotide is administered while the donor patient is undergoing surgery.

In some embodiments, defibrotide is administered to a donor organ. In some embodiments, defibrotide is administered to a donor organ via injection. In some embodiments, defibrotide is administered to a donor organ via injection into an artery or vein.

In some embodiments, defibrotide is administered to a preservation bath that is used to store a donor organ for transfer. As used herein, “preservation bath” is interchangeable with “organ preservation solution.”

In some embodiments, the preservation bath does not contain defibrotide.

In some embodiments, defibrotide is present in the organ preservation solution at a concentration from about 0.5 mM to about 1 M. In some embodiments, a vitamin solute or electrolyte is present in the organ preservation solution at a concentration of about 0.5 mM, about 1.0 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, about 50 mM, about 51 mM, about 52 mM, about 53 mM, about 54 mM, about 55 mM, about 56 mM, about 57 mM, about 58 mM, about 59 mM, about 60 mM, about 61 mM, about 62 mM, about 63 mM, about 64 mM, about 65 mM, about 66 mM, about 67 mM, about 68 mM, about 69 mM, about 70 mM, about 71 mM, about 72 mM, about 73 mM, about 74 mM, about 75 mM, about 76 mM, about 77 mM, about 78 mM, about 79 mM, about 80 mM, about 81 mM, about 82 mM, about 83 mM, about 84 mM, about 85 mM, about 86 mM, about 87 mM, about 88 mM, about 89 mM, about 90 mM, about 91 mM, about 92 mM, about 93 mM, about 94 mM, about 95 mM, about 96 mM, about 97 mM, about 98 mM, about 99 mM, about 100 mM, about 101 mM, about 102 mM, about 103 mM, about 104 mM, 105 mM, about 106 mM, about 107 mM, about 108 mM, about 109 mM, about 110 mM, about 111 mM, about 112 mM, about 113 mM, about 114 mM, about 115 mM, about 116 mM, about 117 mM, about 118 mM, about 119 mM, about 120 mM, about 121 mM, about 122 mM, about 123 mM, about 124 mM, about 125 mM, about 126 mM, about 127 mM, about 128 mM, about 129 mM, about 130 mM, about 131 mM, about 132 mM, about 133 mM, about 134 mM, about 135 mM, about 136 mM, about 137 mM, about 138 mM, about 139 mM, about 140 mM, about 141 mM, about 142 mM, about 143 mM, about 144 mM, about 145 mM, about 146 mM, about 147 mM, about 148 mM, about 149 mM, about 150 mM, about 151 mM, about 152 mM, about 153 mM, about 154 mM, about 155 mM, about 156 mM, about 157 mM, about 158 mM, about 159 mM, about 160 mM, about 161 mM, about 162 mM, about 163 mM, about 164 mM, about 165 mM, about 166 mM, about 167 mM, about 168 mM, about 169 mM, about 170 mM, about 171 mM, about 172 mM, about 173 mM, about 174 mM, about 175 mM, about 176 mM, about 177 mM, about 178 mM, about 179 mM, about 180 mM, about 181 mM, about 182 mM, about 183 mM, about 184 mM, about 185 mM, about 186 mM, about 187 mM, about 188 mM, about 189 mM, about 190 mM, about 191 mM, about 192 mM, about 193 mM, about 194 mM, about 195 mM, about 196 mM, about 197 mM, about 198 mM, about 199 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 450 mM, about 500 mM, about 550 mM, about 600 mM, about 650 mM, about 700 mM, about 750 mM, about 800 mM, about 850 mM, about 900 mM, about 950 mM, or about 1 M, including all values and ranges in between. In some embodiments, defibrotide is present in the organ preservation solution at a concentration of about 2.2 mM. In some embodiments, defibrotide is present in the organ preservation solution at a concentration of about 14.07 mM.

In some embodiments, a donor organ is stored in a preservation bath optionally comprising defibrotide at a temperature from about 0° C. to about 50° C. In some embodiments, a donor organ is stored in a preservation bath optionally comprising defibrotide at a temperature of about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., or about 50° C.

In some embodiments, an organ preservation solution optionally comprising defibrotide has a colloidal osmotic pressure of between about 0 mmHg and about 50 mmHg. In some embodiments, the colloidal osmotic pressure is about 0 mmHg, about 1 mmHg, about 2 mmHg, about 3 mmHg, about 4 mmHg, about 5 mmHg, about 6 mmHg, about 7 mmHg, about 8 mmHg, about 9 mmHg, about 10 mmHg, about 11 mmHg, about 12 mmHg, about 13 mmHg, about 14 mmHg, about 15 mmHg, about 16 mmHg, about 17 mmHg, about 18 mmHg, about 19 mmHg, about 20 mmHg, about 21 mmHg, about 22 mmHg, about 23 mmHg, about 24 mmHg, about 25 mmHg, about 26 mmHg, about 27 mmHg, about 28 mmHg, about 29 mmHg, about 30 mmHg, about 31 mmHg, about 32 mmHg, about 33 mmHg, about 34 mmHg, about 35 mmHg, about 36 mmHg, about 37 mmHg, about 38 mmHg, about 39 mmHg, about 40 mmHg, about 41 mmHg, about 42 mmHg, about 43 mmHg, about 44 mmHg, about 45 mmHg, about 46 mmHg, about 47 mmHg, about 48 mmHg, about 49 mmHg, or about 50 mmHg.

In some embodiments, an organ preservation solution optionally comprising defibrotide has a viscosity of between about 0.5 cP and about 10 cP. In some embodiments, the viscosity is about 0.5 cP, about 0.7 cP, about 0.8 cP, about 0.9 cP, about 1.0 cP, about 1.5 cP, about 2.0 cP, about 2.5 cP, about 3.0 cP, about 3.5 cP, about 4.0 cP, about 4.5 cP, about 5.0 cP, about 5.5 cP, about 6.0 cP, about 6.5 cP, about 7.0 cP, about 7.5 cP, about 8.0 cP, about 8.5 cP, about 9.0 cP, about 9.5 cP, or about 10 cP.

In some embodiments, an organ preservation solution optionally comprising defibrotide has an osmolality of between about 250 mOsm/kg H₂O and 350 mOsm/kg H₂O. In some embodiments, the osmolality is about 250 mOsm/kg H₂O, about 260 mOsm/kg H₂O, about 270 mOsm/kg H₂O, about 280 mOsm/kg H₂O, about 290 mOsm/kg H₂O, about 300 mOsm/kg H₂O, about 310 mOsm/kg H₂O, about 320 mOsm/kg H₂O, about 330 mOsm/kg H₂O, about 340 mOsm/kg H₂O, or about 350 mOsm/kg H₂O.

In some embodiments, an organ preservation solution optionally comprising defibrotide has a colloidal osmotic pressure between about 1 mmHg and 2 mmHg, an osmolality of between about 275 mOsm/kg H₂O and 325 mOsm/kg H₂O, and a viscosity between about 1.2 cP and about 2.0 cP. In some embodiments, an organ preservation solution optionally comprising defibrotide has a colloidal osmotic pressure between about 25 mmHg and 35 mmHg, an osmolality of between about 300 mOsm/kg H₂O and 350 mOsm/kg H₂O, and a viscosity between about 4.5 cP and about 5.5 cP. In some embodiments, an organ preservation solution optionally comprising defibrotide has a colloidal osmotic pressure between about 5 mmHg and 15 mmHg, an osmolality of between about 275 mOsm/kg H₂O and 325 mOsm/kg H₂O, and a viscosity between about 1.6 cP and about 3.6 cP. In some embodiments, an organ preservation solution optionally comprising defibrotide has a colloidal osmotic pressure between about 3 mmHg and 5 mmHg, an osmolality of between about 275 mOsm/kg H₂O and 325 mOsm/kg H₂O, and a viscosity between about 1.2 cP and about 2.0 cP.

In some embodiments, an organ preservation solution optionally comprising defibrotide has a pH between about 3 and about 9. In some embodiments, the organ preservation optionally comprising defibrotide has a pH between about 5.5 and about 8.5. In some embodiments, the organ preservation optionally comprising defibrotide has a pH between about 6 and about 8. In some embodiments, the pH is between about 7 and about 8. In some embodiments, an organ preservation solution optionally comprising defibrotide has a pH of about 3, about 3.5, about 4.0, about 4.5, about 5.0, about 5.5, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.5, or about 9.0

In some embodiments, an organ preservation solution, optionally comprising defibrotide, contains one or more of a lipid, a growth factor, hormone, vitamin, solute, and electrolyte. In some embodiments, a vitamin, solute, or electrolyte is present in the organ preservation solution at a concentration from about 0.5 mM to about 400 mM. In some embodiments, a vitamin solute or electrolyte is present in the organ preservation solution at a concentration of about 0.5 mM, about 1.0 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, about 50 mM, about 51 mM, about 52 mM, about 53 mM, about 54 mM, about 55 mM, about 56 mM, about 57 mM, about 58 mM, about 59 mM, about 60 mM, about 61 mM, about 62 mM, about 63 mM, about 64 mM, about 65 mM, about 66 mM, about 67 mM, about 68 mM, about 69 mM, about 70 mM, about 71 mM, about 72 mM, about 73 mM, about 74 mM, about 75 mM, about 76 mM, about 77 mM, about 78 mM, about 79 mM, about 80 mM, about 81 mM, about 82 mM, about 83 mM, about 84 mM, about 85 mM, about 86 mM, about 87 mM, about 88 mM, about 89 mM, about 90 mM, about 91 mM, about 92 mM, about 93 mM, about 94 mM, about 95 mM, about 96 mM, about 97 mM, about 98 mM, about 99 mM, about 100 mM, about 101 mM, about 102 mM, about 103 mM, about 104 mM, 105 mM, about 106 mM, about 107 mM, about 108 mM, about 109 mM, about 110 mM, about 111 mM, about 112 mM, about 113 mM, about 114 mM, about 115 mM, about 116 mM, about 117 mM, about 118 mM, about 119 mM, about 120 mM, about 121 mM, about 122 mM, about 123 mM, about 124 mM, about 125 mM, about 126 mM, about 127 mM, about 128 mM, about 129 mM, about 130 mM, about 131 mM, about 132 mM, about 133 mM, about 134 mM, about 135 mM, about 136 mM, about 137 mM, about 138 mM, about 139 mM, about 140 mM, about 141 mM, about 142 mM, about 143 mM, about 144 mM, about 145 mM, about 146 mM, about 147 mM, about 148 mM, about 149 mM, about 150 mM, about 151 mM, about 152 mM, about 153 mM, about 154 mM, about 155 mM, about 156 mM, about 157 mM, about 158 mM, about 159 mM, about 160 mM, about 161 mM, about 162 mM, about 163 mM, about 164 mM, about 165 mM, about 166 mM, about 167 mM, about 168 mM, about 169 mM, about 170 mM, about 171 mM, about 172 mM, about 173 mM, about 174 mM, about 175 mM, about 176 mM, about 177 mM, about 178 mM, about 179 mM, about 180 mM, about 181 mM, about 182 mM, about 183 mM, about 184 mM, about 185 mM, about 186 mM, about 187 mM, about 188 mM, about 189 mM, about 190 mM, about 191 mM, about 192 mM, about 193 mM, about 194 mM, about 195 mM, about 196 mM, about 197 mM, about 198 mM, about 199 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, or about 400 mM, including all values and ranges in between.

As used herein, an electrolyte in an organ preservation solution, optionally comprising defibrotide, comprises an anion and/or cation. In some embodiments, the electrolyte is a salt. In some embodiments, a cation is selected from the group consisting of calcium, magnesium, potassium, and sodium. In some embodiments, an organ preservation solution optionally comprising defibrotide contains sodium, potassium, and magnesium. In some embodiments, the organ preservation solution optionally comprising defibrotide contains sodium, potassium, magnesium, and calcium.

In some embodiments, an anion in an organ preservation bath, optionally comprising defibrotide, comprises a carboxylate and/or hydroxyl group. In certain embodiments, the anion is selected from the group consisting of acetate, citrate, lactate, aspartate, sorbate, ascorbate, and propionate. In some embodiments, an anion is inorganic and is selected from the group consisting of phosphate, sulfate, chloride, hydroxide, carbonate, bicarbonate, bisulfite, and metabisulfite. In some embodiments, an electrolyte is selected from the group consisting of calcium acetate, calcium citrate, calcium lactate, calcium phosphate, calcium sulfate, calcium sulfate dihydrate, magnesium acetate, magnesium aspartate, magnesium chloride, magnesium hydroxide, magnesium phosphate, magnesium sulfate, potassium chloride, potassium citrate, potassium phosphate monobasic, potassium phosphate dibasic, potassium sorbate, sodium acetate, sodium ascorbate, sodium bicarbonate, sodium bisulfite, sodium chloride, sodium citrate, sodium hydroxide, sodium lactate, sodium metabisulfite, sodium phosphate monobasic, sodium phosphate dibasic, sodium propionate, and sodium sulfate.

In some embodiments, a vitamin is added to the organ preservation solution, optionally comprising defibrotide. Non-limiting examples of vitamins include vitamin A, vitamin B1, vitamin B2, vitamin B6, vitamin B12, vitamin C, vitamin D, vitamin K, vitamin E, biotin, folic acid, pantothenic acid, niacin, m-inositol, calcium, phosphorus, magnesium, zinc, manganese, copper, sodium, potassium, chloride, iron and selenium.

In some embodiments, an organ preservation solution, optionally comprising defibrotide, contains a buffer. Non-limiting examples of buffers include histidine, glycine, phosphate, tryptophan, BES, MES, TAPS, MOPS, HEPES, or Tris buffer. BES, MES, TAPS, and MOPS are zwitterionic buffering agents. In some embodiments, the organ preservation solution contains histidine, glycine, and tryptophan buffers. In some embodiments, the organ preservation solution contains glycine and BES buffers.

In some embodiments, an organ preservation solution, optionally comprising defibrotide, contains one or more impermeants. Non-limiting examples of impermeants include glucose, raffinose, sucrose, colloids, hydroxyethyl starch (HES), and polyethylene glycol (PEG). The PEG may have a molecular weight from about 300 g/mol to about 10,000,000 g/mol. Non-limiting examples of PEGS include PEG400, PEG1500, PEG3350, PEG4000, PEG6000, PEG8000, and PEG35000.

In some embodiments, an organ preservation solution, optionally comprising defibrotide, contains one or more amino acids. Non-limiting examples of amino acids include the L- and/or D- enantiomers of glutamine, asparagine, glutamic acid, aspartic acid, serine, threonine, methionine, glycine, proline, phenylalanine, alanine, methionine, isoleucine, leucine, tyrosine, valine, tryptophan, histidine, lysine, and arginine.

In some embodiments, an organ preservation solution, optionally comprising defibrotide, contains one or more of adenosine, glutathione, N-acetyl histidine, allopurinol, alpha-ketoglutarate, aspartate, ordeferoxamine/L20 iron chelator. In some embodiments, an organ preservation solution comprises saline, Ringer's solution, Celsior solution, Kyoto University solution, IGL-1, Tyrode's solution, histidine-tryptophan-ketoglutarate (HTK), Steen Solution™, Viaspan®, BGP-CS, BGP-HMP, Euro-Collins solution, Collins solution, University of Washington solution, and/or Organ Care System perfusate.

In some embodiments, a donor organ is preserved using standard cold storage. In some embodiments, a donor organ which undergoes standard cold storage is stored on ice after removal from the donor and removed from the ice before the transplant. In some embodiments, the donor organ is removed from ice from about 24 hours to about 30 seconds before transplant, for example, about 24 hours, about 23 hours, about 22 hours, about 21 hours, about 20 hours, about 19 hours, about 18 hours, about 17 hours, about 16 hours, about 15 hours, about 14 hours, about 13 hours, about 12 hours, about 11 hours, about 10 hours, about 9 hours, about 8 hours, about 7 hours, about 6 hours, about 5 hours, about 4 hours, about 3 hours, about 2 hours, about 1 hour, about 45 minutes, about 30 minutes, about 29 minutes, about 28 minutes, about 27 minutes, about 26 minutes, about 25 minutes, about 24 minutes, about 23 minutes, about 22 minutes, about 21 minutes, about 20 minutes, about 19 minutes, about 18 minutes, about 17 minutes, about 16 minutes, about 15 minutes, about 14 minutes, about 13 minutes, about 12 minutes, about 11 minutes, about 10 minutes, about 9 minutes, about 8 minutes, about 7 minutes, about 6 minutes, about 5 minutes, about 4 minutes, about 3 minutes, about 2 minutes, about 1 minute, or about 30 seconds before transplant.

In some embodiments, a donor organ which undergoes standard cold storage is flushed with an organ preservation solution optionally comprising defibrotide at about 0° C. to about 10° C. and immersed in an organ preservation solution until transplantation. In some embodiments, a donor organ which undergoes standard cold storage is flushed with an organ preservation solution optionally comprising defibrotide at about 0° C. to about 4° C. and immersed in an organ preservation solution until transplantation. In some embodiments, after being flushed, the donor organ is stored in an organ preservation bath optionally comprising defibrotide for about 30 seconds to about 24 hours, for example about 30 seconds, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours.

In some embodiments, a donor organ is perfused (e.g. blood or organ preservation solution is flowed through the donor organ). In some embodiments, a donor organ is perfused using an organ preservation solution optionally comprising defibrotide. In some embodiments, a machine is used to perfuse the donor organ. In some other embodiments, a human manually perfuses the donor organ using a syringe or other instrument. In some embodiments, a donor organ is stored in an organ preservation solution that does not contain defibrotide and is not perfused.

In some embodiments, a donor organ is stored in an organ preservation solution comprising defibrotide and is not perfused.

In some embodiments, a donor organ is stored in an organ preservation solution comprising defibrotide and is perfused using an organ preservation solution that does not comprise defibrotide.

In some embodiments, a donor organ is stored in an organ preservation solution comprising defibrotide and is perfused using an organ preservation solution that comprises defibrotide.

In some embodiments, a donor organ is stored in an organ preservation solution that does not contain defibrotide and is perfused using an organ preservation solution that does not comprise defibrotide.

In some embodiments, a donor organ is stored in an organ preservation solution that does not contain defibrotide and is perfused using an organ preservation solution that comprises defibrotide.

In some embodiments, a donor organ is stored in an organ preservation solution optionally comprising defibrotide and is perfused. In some embodiments, the organ preservation solution used for perfusion contains defibrotide.

In some embodiments, a donor organ is stored via static cold storage and is perfused. In some embodiments, the organ preservation solution used for perfusion comprises defibrotide.

In some embodiments, perfusion of a donor organ occurs before, during, or after organ transplant. In some embodiments, a donor organ is perfused with an organ preservation solution, optionally containing defibrotide, and stored in an organ preservation solution, optionally containing defibrotide.

In some embodiments, the donor organ is perfused with blood. In some embodiments, the donor organ is perfused with an organ preservation solution, optionally containing defibrotide. In some embodiments, the donor organ is perfused with an organ preservation solution optionally containing defibrotide and blood.

In some embodiments, organ perfusion occurs at a temperature from about 0° C. to about 50° C. In some embodiments, perfusion with an organ perfusion solution optionally containing defibrotide occurs at a temperature of about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., or about 50° C.

In some embodiments, perfusion is hypothermic, e.g. perfusion occurs at a temperature from about 0° C. to about 12° C. In some embodiments, hypothermic perfusion occurs at about 0° C., about 1° C., about 2° C., about 3° C., about 4° C., about 5° C., about 6° C., about 7° C., about 8° C., about 9° C., about 10° C., about 11° C., or about 12° C. In some embodiments, perfusion is performed using midthermic machine perfusion, e.g. perfusion occurs at a temperature from about 13° C. to about 24° C. In some embodiments, midthermic machine perfusion occurs at a temperature of about 13° C., about 14° C., about 15° C., about 16° C., about 17° C., about 18° C., about 19° C., about 20° C., about 21° C., about 22° C., about 23° C., or about 24° C. In some embodiments, perfusion is performed using subnormothermic machine perfusion, e.g. perfusion occurs at a temperature from about 25° C. to about 34° C. In some embodiments, subnormothermic machine perfusion occurs at a temperature of about 25° C., about 26° C., about 27° C., about 28° C., about 29° C., about 30° C., about 31° C., about 32° C., about 33° C., or about 34° C. In some embodiments, perfusion is performed using normothermic machine perfusion, e.g. perfusion occurs at a temperature from about 35° C. to about 38° C. In some embodiments, normothermic machine perfusion occurs at a temperature of about 35° C., about 36° C., about 37° C., or about 38° C.

In some embodiments, defibrotide is administered to a patient undergoing, about to undergo, or having received an organ transplant. In some embodiments, defibrotide is administered parenterally to a patient undergoing, about to undergo, or having received an organ transplant. In some embodiments, defibrotide is administered to a patient undergoing, about to undergo, or having received an organ transplant intravenously, intramuscularly, subcutaneously, or transdermally. In some embodiments, defibrotide is administered to a patient undergoing, about to undergo, or having received an organ transplant by injection. In some embodiments, defibrotide is administered to a patient undergoing, about to undergo, or having received an organ transplant by injection into an artery or a vein.

In some embodiments, defibrotide may be administered:
a. to a patient undergoing, about to undergo, or having received an organ transplant and
b. to a donor organ .

In some embodiments, defibrotide may be administered:
a. to a patient undergoing, about to undergo, or having received an organ transplant and
b. to a preservation bath that is used to store a donor organ for transfer.

In some embodiments, defibrotide may be administered:
a. to a patient undergoing, about to undergo, or having received an organ transplant,
b. to a donor organ , and
c. to a preservation bath that is used to store a donor organ for transfer.

In some embodiments, defibrotide may be administered:
a. to a donor organ , and
b. to a preservation bath that is used to store a donor organ for transfer.

In some embodiments, a donor organ for transfer is stored in a preservation bath that optionally contains defibrotide for about 30 seconds to about 48 hours before transplantation. In some embodiments, a donor organ for transfer is stored in a preservation bath that contains defibrotide for about 30 seconds to about 10 minutes before transplantation. In some embodiments, a donor organ for transfer is stored in a preservation bath that optionally contains defibrotide for about 10 minutes to 2 hours before transplantation. In some embodiments, a donor organ for transfer is stored in a preservation bath that optionally contains defibrotide for about 30 seconds, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 inutes, about 28 minutes, about 29 minutes, about 30 minutes, about 31 minutes, 32 minutes, 33 minutes, 34 minutes, 35 minutes, 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, about 12 hours, about 12.5 hours, about 13 hours, about 13.5 hours, about 14 hours, about 14.5 hours, about 15 hours, about 15.5 hours, about 16 hours, about 16.5 hours, about 17 hours, about 17.5 hours, about 18 hours, about 18.5 hours, about 19 hours, about 19.5 hours, about 20 hours, about 20.5 hours, about 21 hours, about 21.5 hours, about 22 hours, about 22.5 hours, about 23 hours, about 23. 5 hours, about 24 hours, about 24.5 hours, about 25 hours, about 25.5 hours, about 26 hours, about 26.5 hours, about 27 hours, about 27.5 hours, about 28 hours, about 28.5 hours, about 29 hours, about 29.5 hours, about 30 hours, about 30.5 hours, about 31 hours, about 31.5 hours, about 32 hours, about 32.5 hours, about 33 hours, about 33.5 hours, about 34 hours, about 34. 5 hours, about 35 hours, about 35.5 hours, about 36 hours, about 36.5 hours, about 37 hours, about 37.5 hours, about 38 hours, about 38.5 hours, about 39 hours, about 39.5 hours, about 40 hours, about 40.5 hours, about 41 hours, about 41.5 hours, about 42 hours, about 42.5 hours, about 43 hours, about 43.5 hours, about 44 hours, about 44.5 hours, about 45 hours, about 45.5 hours, about 46 hours, about 46.5 hours, about 47 hours, about 47.5 hours, or about 48 hours before transplantation.

As a skilled artisan will appreciate, a defibrotide treatment period may vary on a patient-by-patient basis. In some embodiments, a defibrotide treatment period may vary depending on the assessed likelihood of organ injury and/or organ rejection. In some embodiments, a defibrotide treatment period is determined by monitoring signs and symptoms of organ injury and/or organ rejection. For example, if the signs and symptoms of organ injury or rejection are still present after an initial treatment period, defibrotide treatment is continued until resolution of organ injury or rejection. As a skilled artisan will appreciate, a defibrotide treatment period may vary on a patient-by-patient basis. In some embodiments, a defibrotide treatment period is determined by monitoring signs and symptoms of organ injury and/or organ rejection or consequences thereof. For example, if the signs and symptoms of organ injury and/or organ rejection or consequences thereof are still present after an initial treatment period, defibrotide treatment is continued until resolution of organ injury and/or organ rejection or consequences thereof

In some embodiments, a treatment period lasts from about 1 day to about 1 year, for example about 1 day, 2 days, 3 days, 4 days, 5 days, 6 days, 7 days, 8 days, 9 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 6 weeks, 2 months, 3 months, 4 months, 5 months, 6 months, 7 months, 8 months, 9 months, 10 months, 11 months, or 12 months, or more, including all values and ranges in between thereof. In some embodiments, a treatment period lasts 1 week.

The defibrotide dosing may be determined by a variety of factors that will be readily apparent to a skilled artisan. In some embodiments, a dose is based on patient's baseline body weight, defined as the patient's weight prior to organ transplant. In some embodiments, defibrotide is administered in an amount of about 1 to about 100 mg/kg of body weight per day. For example defibrotide is administered in an amount of about 1 mg/kg, about 1.25 mg/kg, about 1.50 mg/kg, about 1.75 mg/kg, about 2 mg/kg, about 2.25 mg/kg, about 2.50 mg/kg, about 2.75 mg/kg, about 3 mg/kg, about 3.25 mg/kg, about 3.50 mg/kg, about 3.75 mg/kg, about 4.25 mg/kg, about 4.50 mg/kg, about 4.75 mg/kg, about 5 mg/kg, about 5.25 mg/kg, about 5.50 mg/kg, about 5.75 mg/kg, about 6 mg/kg, about 6.25 mg/kg, about 6.50 mg/kg, about 6.75 mg/kg, about 7 mg/kg, about 7.25 mg/kg, about 7.50 mg/kg, about 7.75 mg/kg, about 8 mg/kg, about 8.25 mg/kg, about 8.50 mg/kg, about 8.75 mg/kg, about 9 mg/kg, about 9.25 mg/kg, about 9.50 mg/kg, about 9.75 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg of body weight per day, including all ranges therebetween. In some embodiments, defibrotide is administered in an amount of about 25 mg per kilogram of body weight per day. In some embodiments, doses based on the patient's body weight were rounded to the nearest 10 mg for patients over 35 kg. In some embodiments, doses based on the patient's body weight were rounded to the nearest 5 mg for patients under 35 kg. In some embodiments, defibrotide is administered as a high concentration low viscosity formulation, as described in WO 2019/028340 the contents of which are incorporated by reference in their entirety for all purposes.

The defibrotide may be administered as a single daily dose or in multiple doses per day. In some embodiments, defibrotide is administered once a day. In some embodiments, defibrotide is administered in multiple doses per day. For example, defibrotide may be administered in 2, 3, 4, 5, 6, 7, 8, 9, or in 10 doses per day. In some embodiments, defibrotide is administered in four doses per day. In some embodiments, defibrotide is administered in four doses per day every 6 hours.

In some embodiments, when defibrotide is administered in multiple doses per day, the different doses of defibrotide are administered from about 30 minutes to about 12 hours apart. In some embodiments, defibrotide is administered about every 30 minutes, about every 40 minutes, about every 50 minutes, about every 60 minutes, about every 70 minutes, about every 80 minutes, about every 90 minutes, about every 2 hours, about every 3 hours, about every 4 hours, about every 5 hours, about every 6 hours, about every 7 hours, about every 8 hours, about every 9 hours, about every 10 hours, about every 11 hours, or about every 12 hours. In some embodiments, when defibrotide is administered in multiple doses per day, the different doses of defibrotide are administered about 6 hours apart.

The defibrotide may be administered daily, weekly, or monthly. In some embodiments, the defibrotide is administered every day for about one week, about two weeks, about three weeks, or about four weeks, or more. In some embodiments, defibrotide administration occurs on consecutive days. In some embodiments, defibrotide administration occurs on discontinuous days.

In some embodiments, defibrotide administration begins on the day of organ transplant. In some embodiments, defibrotide administration begins before transplant surgery commences. In some embodiments, defibrotide administration begins during transplant surgery. In some embodiments, defibrotide administration begins after transplant surgery.

In some embodiments, defibrotide administration occurs between about 1 day and 1 month or more before organ transplant. In some embodiments, defibrotide administration occurs before organ transplant, for example, about 1 day, about 2 days, about 3 days, about 4 days, about 5 days, about 6 days, about 7 days, about 8 days, about 9 days, about 10 days, about 11 days, about 12 days, about 13 days, about 14 days, about 3 weeks, about 4 weeks, or about 1 month or more before organ transplant.

In certain embodiments, defibrotide is administered prophylactically to a patient to prevent organ injury and/or organ rejection. In some embodiments, one or more administrations of the defibrotide begin before organ transplant. In some embodiments, one or more defibrotide treatments begin before the patient is diagnosed with organ injury and/or organ rejection, or experiences consequences of organ injury and/or organ rejection.

In some embodiments, defibrotide administration occurs between about 1 hour and 1 month after organ transplant, for example, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 1 day, about 2 days, about 3 days, about 4 days, about 5 days, about 6 days, about 7 days, about 8 days, about 9 days, about 10 ays, about 11 days, about 12 days, about 13 days, about 14 days, about 3 weeks, about 4 weeks, or about 1 month or more after organ transplant. In some embodiments, defibrotide administration begins six hours after organ transplant.

In some embodiments, defibrotide administration occurs before development of ischemia reperfusion injury. In some embodiments, one or more administrations of defibrotide are administered to treat or prevent ischemia reperfusion injury. In some embodiments, defibrotide administration occurs before development of ischemia reperfusion injury in a patient who is at risk of developing the same. In some embodiments, defibrotide administration occurs after the development of ischemia reperfusion injury. In some embodiments, defibrotide is administered until symptoms improve. In some embodiments, defibrotide is administered until symptoms are eradicated. In some embodiments, defibrotide is administered until organ injury and/or organ rejection or consequences of organ injury and/or rejection are cured.

The timing of the administration of the defibrotide may depend on the particular patient (e.g. what type of transplant the patient receives; age of patient, etc.) and the patient's medical history.

Any combination of the foregoing embodiments may be used in treating the patient with defibrotide. Accordingly, in some embodiments, a patient is administered defibrotide intravenously in an amount of about 2.5 mg per kilogram of body weight about every 6 hours. Accordingly, in some embodiments, a patient is administered defibrotide intravenously in an amount of about 6.25 mg per kilogram of body weight about every 6 hours.

The defibrotide may be administered by any suitable route, including without limitation parenteral (e.g., intravenous, subcutaneous, intrastemal, intramuscular, or infusion techniques), oral, sublingual, buccal, intranasal, pulmonary, topical, transdermal, intradermal, mucosal, ocular, otic, rectal, vaginal, intragastric, intrasynovial, and intra- articular routes. In some embodiments, defibrotide is administered intravenously. In some embodiments, defibrotide is administered via intravenous infusion. In some embodiments, defibrotide is administered by constant intravenous infusion over a 2-hour period. In some embodiments, the defibrotide is diluted prior to infusion. In some embodiments, the diluted defibrotide solution is administered using an infusion set equipped with a filter (e.g., a 0.2 micron in-line filter). In certain embodiments, the intravenous administration line (e.g., peripheral or central) is flushed immediately before and after administration (e.g., with 5% Dextrose Injection, USP or 0.9% Sodium Chloride Injection, USP).

In some embodiments, the defibrotide is administered subcutaneously. In some embodiments, the defibrotide is administered subcutaneously by means of a device that is commercially available such as, for example, the FREEDOM60® pump or similar (RMSTM Medical Products). In some embodiments, the defibrotide is administered subcutaneously using an automated injection device. Subcutaneous administration of a high concentration low viscosity defibrotide formulation via an automated injection device may offer significant reduction of the time for clinical administration and enable outpatient dosing of the product for as long as needed. The use of an automated injection device improves convenience and allows faster administration by health-care professionals (HCP), care-givers, or even self-administration by the patients.

In some embodiments, the route of administration affects the efficacy and/or longevity of the formulations of the present disclosure. In some embodiments, subcutaneous, intramuscular and/or intraperitoneal administration is associated with an extended systemic half-life compared to the same formulation administered intravenously. In some embodiments, subcutaneous administration of the formulation provides lower peak-to-trough ratios of plasma concentrations compared to the same formulation administered intravenously. In some embodiments, subcutaneous administration provides improved efficacy and/or improves the safety profile of the formulation compared to the same formulation administrated intravenously.

Devices for subcutaneous administration may be prefilled, with for example a predefined adult or pediatric dose, or may be used to administer a weight-based dose specific for individual patients. In some embodiments, the patient determines the dose and administers it. In some specific embodiments, formulations of the disclosure are administered subcutaneously in less than about two hours, less than about one hour, or less than about 30 minutes. In some specific embodiments, formulations of the disclosure are delivered subcutaneously over about 5 minutes to about 1 hour, about 10 minutes to about 1 hour or about 15 minutes to about 45 minutes.

In some embodiments, subcutaneous administration of the low-viscosity formulations of the present disclosure allows for less-frequent administration and/or lower doses. In some embodiments, subcutaneous administration of the low-viscosity formulation of the present disclosure allows for reduced administration volume.

In some embodiments, administration of defibrotide prevents, delays, treats, or ameliorates development of organ injury and/or organ failure or consequences thereof, including acute and chronic graft rejection, delayed graft function, and chronic graft dysfunction compared to an untreated patient or the same patient before defibrotide administration or as compared to a patient that receives an organ transplant without defibrotide administration.

In some embodiments, organ injury and/or organ rejection or consequences thereof, including acute and chronic graft rejection, delayed graft function, and chronic graft dysfunction are prevented, delayed, treated, or ameliorated in the patient between day 1 and year 10.

In some embodiments, administration of defibrotide prevents, delays, treats, or ameliorates development of organ injury and/or organ failure or consequences thereof, including acute and chronic graft rejection, delayed graft function, and chronic graft dysfunction compared to an untreated patient or the same patient before defibrotide administration, or to a patient that receives an organ transplant without defibrotide administration, at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3. In some embodiments, administration of defibrotide prevents, delays, treats, or ameliorates development of organ injury and/or organ failure or consequences thereof, including acute and chronic graft rejection, delayed graft function, and chronic graft dysfunction compared to an untreated patient or the same patient before defibrotide administration or compared to a patient that receives an organ transplant without defibrotide administration for about 1 day, about 1 week, about 1 month, about 2 months, about 3 months, about 4 months, about 5 months, about 6 months, about 1 year, about 2 years, about 5 years, or about 10 years, or more.

In some embodiments, administration of defibrotide prevents, delays, treats, or ameliorates organ injury and/or organ rejection or consequences thereof, including acute and chronic graft rejection, delayed graft function, and chronic graft dysfunction by about 1% to about 100%, for example, about 5%, about 10%, about 20%, about 30%, about 40%, about 50%, about 60%, about 70%, about 80%, about 90%, or about 100% compared to an untreated patient, the same patient before defibrotide administration, or to a patient that receives an organ transplant without administration of defibrotide.

In some embodiments, administration of defibrotide prevents, delays, treats, or ameliorates organ injury and/or organ rejection or symptoms thereof, including acute and chronic graft rejection, delayed graft function, and chronic graft dysfunction, compared to an untreated patient, the same patient before defibrotide administration, or to a patient that receives an organ transplant without administration of defibrotide by about 1% to about 100%, for example, about 1%, about 5%, about 10%, about 20%, about 30%, about 40%, about 50%, about 60%, about 70%, about 80%, about 90%, or about 100%, for about 1 day to 10 years, or more, for example about 1 day, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years or more.

In some embodiments, administration of defibrotide modulates the expression of biomarkers associated with organ injury and/or organ rejection or consequences thereof, including acute and chronic graft rejection, delayed graft function, and chronic graft dysfunction compared to an untreated or the same patient before defibrotide administration or to a patient that receives a transplant without defibrotide administration.

In some embodiments, administration of the defibrotide decreases expression levels of biomarkers associated with organ injury and/or organ rejection or consequences thereof, including acute and chronic graft rejection, delayed graft function, and chronic graft dysfunction compared to an untreated patient or the same patient before organ transplant or to a patient that receives an organ transplant without defibrotide administration, or a related disorder. In some embodiments, biomarker expression is decreased by about 5% to about 100%, for example, about 5%, about 10%, about 15%, about 20%, about 25%, about 30%, about 35%, about 40%, about 45%, about 50%, about 55%, about 60%, about 65%, about 70%, about 80%, about 85%, about 90%, about 95%, or about 100%, compared to prior to defibrotide administration or compared to a patient that received an organ transplant without defibrotide administration.

In some embodiments, administration of defibrotide modulates expression of one or more urine biomarkers, selected from cystatin-C, neutrophil gelatinase-associated lipocalin (NGAL), monocyte chemoattractant protein-1 (MCP-1), Interleukin 18 (IL-18), kidney injury molecule-1 (KIM-1), beta-2-microglobulin (B2M), urine total protein, and urinary microalbumin. In some embodiments, urine biomarker expression is increased by about 5% to about 100%, for example about 5%, about 10%, about 15%, about 20%, about 25%, about 30%, about 35%, about 40%, about 45%, about 50%, about 55%, about 60%, about 65%, about 70%, about 80%, about 85%, about 90%, about 95%, or about 100%, compared to prior to defibrotide administration or compared to a patient that received an organ transplant without defibrotide administration. In some embodiments, urine biomarker expression is decreased by about 5% to about 100%, for example, about 5%, about 10%, about 15%, about 20%, about 25%, about 30%, about 35%, about 40%, about 45%, about 50%, about 55%, about 60%, about 65%, about 70%, about 80%, about 85%, about 90%, about 95%, or about 100%, compared to prior to defibrotide administration or compared to a patient that received an organ transplant without defibrotide administration.

In some embodiments, administration of defibrotide modulates one or more serum biomarkers selected from the group consisting of C-reactive protein (CRP), endothelin 1 (ET-1), serum adhesion molecule-1 (ICAM-1), ICAM, E-Selectin, tissue plasminogen activator (t-PA), plasminogen activator inhibitor type 1 (PAI-1), PAI, Thrombomodulin, interleukin 6 (IL-6), creatinine, tumor necrosis factor alpha (TNF-α), or monocyte chemoattractant protein-1 (MCP-1). In some embodiments, serum biomarker expression is increased by about 5% to about 100%, for example, about 5%, about 10%, about 15%, about 20%, about 25%, about 30%, about 35%, about 40%, about 45%, about 50%, about 55%, about 60%, about 65%, about 70%, about 80%, about 85%, about 90%, about 95%, or about 100%, compared to prior to defibrotide administration or compared to a patient that received an organ transplant without defibrotide administration. In some embodiments, serum biomarker expression is decreased by about 5%, about 10%, about 15%, about 20%, about 25%, about 30%, about 35%, about 40%, about 45%, about 50%, about 55%, about 60%, about 65%, about 70%, about 80%, about 85%, about 90%, about 95%, or about 100%, compared to prior to defibrotide administration or compared to a patient that received an organ transplant without defibrotide administration.

In some embodiments, biomarkers expression associated with organ injury and/or organ rejection or consequences thereof, including acute and chronic graft rejection, delayed graft function, and chronic graft dysfunction is modulated in the patient between day 1 and year 10 compared to an untreated patient or the same patient before defibrotide administration or to a different patient that has received an organ transplant without defibrotide administration, for example, at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3. In some embodiments, administration of defibrotide modulates the expression of biomarkers associated with organ injury and/or organ rejection or consequences thereof, including acute and chronic graft rejection, delayed graft function, and chronic graft dysfunction compared to an untreated patient or the same patient before defibrotide administration or to a patient that has received an organ transplant without defibrotide administration for about 1 day to about 10 years, or more, for example, 1 day, about 1 week, about 1 month, about 2 months, about 3 months, about 4 months, about 5 months, about 6 months, about 1 year, about 2 years, about 5 years, or about 10 years.

In accordance with some embodiments of the present disclosure, a patient is from about 0 years of age to about 16 years of age, including all ranges and subranges therein. For example, a patient is from about 0 months, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, to about 16 years of age. In some embodiments, a patient is from about 0 months to about 23 months of age. In some embodiments, a patient is from about 2 years to about 11 years of age. In some embodiments, a patient is from about 12 years to about 16 years of age. In accordance with some embodiments of the present disclosure, a patient may be a pediatric patient or adult. A pediatric patient is from about 0 years of age to about 16 years of age, including all ranges and subranges therein. For example, a pediatric patient is from about 0 months, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, to about 16 years of age. In some embodiments, a patient is from about 0 months to about 23 months of age. In some embodiments, a patient is from about 2 years to about 11 years of age. In some embodiments, a patient is from about 12 years to about 16 years of age.

In some embodiments, a patient is an adult patient. An adult patient is older than 16 years of age. In some embodiments, the adult patient is 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, or 105 years of age. In some embodiments, an adult patient is between 16 and 30 years of age, including all values and ranges in between. In some embodiments, an adult patient is between 31 and 40 years of age, including all values and ranges in between. In some embodiments, an adult patient is between 41 and 50 years of age, including all values and ranges in between. In some embodiments, an adult patient is between 51 and 60 years of age, including all values and ranges in between. In some embodiments, an adult patient is between 61 and 70 years of age, including all values and ranges in between. In some embodiments, an adult patient is between 71 and 80 years of age, including all values and ranges in between. In some embodiments, an adult patient is between 81 and 90 years of age, including all values and ranges in between. In some embodiments, thane adult patient is between 91 and 100 years of age, including all values and ranges in between.

In some embodiments, a patient of the present disclosure may have organ injury and/or organ rejection, or consequences thereof. Non-limiting examples of organ injuries include interstitial fibrosis, tubular atrophy, glomerulosclerosis, acute kidney injury, diffuse alveolar damage, nonalcoholic steatohepatitis, fibrosis, cardiac allograph vasculopathy, or ischemia reperfusion injury (IRI). In some embodiments, a patient of the present disclosure may have a consequence of organ rejection, including delayed graft function (DGF), graft dysfunction, chronic lung allograft dysfunction (CLAD), acute allograft dysfunction, chronic allograft dysfunction, primary graft non-function, and primary graft failure (PGF). In some embodiments, the organ injury is IRI.

In some embodiments, a patient of the present disclosure may have or be receiving any organ transplant, graft, or allograft. In some embodiments, a donor organ is selected from pancreas, bowel, small bowel, heart, heart valve, stomach, testis, liver, lungs, kidneys, pancreas, uterus, intestine, bladder, composite tissue, xenograft, and thymus. In some embodiments, the patient of the present disclosure may have or be receiving a transplant of tissues (e.g. donor tissue) including bones, bone marrow, tendons, corneae, skin, valves, nerves, and veins. As a result of transplant, a patient is at risk for organ injury and/or organ rejection or consequences thereof, including acute and chronic graft rejection, delayed graft function, acute graft dysfunction, and chronic graft dysfunction.

In some embodiments, a donor organ is obtained from a live donor. In some embodiments, a donor organ is obtained from a deceased donor. In some embodiments, the deceased donor organ is obtained from a decreased patient after cardiac death (e.g. a donation after cardiac death (DCD)) or a deceased patient after brain death (e.g. donation after brain death (DBD)).

In some embodiments, donor organs have marginal characteristics. Non-limiting examples of marginal characteristics include age greater than 55 years, hepatitis C positive, cocaine use, ejection fraction less than 0.45, or donor to recipient body mass index mismatch of greater than 20%. In some embodiments, the donor organ is from a donor that is between about 55 years old and 100 years old, for example, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, or about 100 years old.

In some embodiments, marginal characteristics include, but are not limited to, organs obtained from expanded criteria donors (ECD) (e.g. donors that were 60 years or older of age or aged 50-59 with 2 additional risk factors [e.g. history of hypertension, death as a result of cerebrovascular accident, or an elevated terminal creatinine]).

In some embodiments, donated organs have organ injury and/or organ rejection. In some embodiments, organ injury and/or organ rejection is a result of donor management, organ recovery, storage, implantation, reperfusion, and from recipient-related factors.

In some embodiments, the patients of the disclosure have any disease that requires or benefits from organ transplantation. Non-limiting examples of diseases include cardiomyopathy, cirrhosis, chronic obstructive pulmonary disease, coronary heart disease, cystic fibrosis, diabetes, hepatitis, cancer, hypertension, kidney disease, causes leading to end stage kidney failure, end stage liver failure, end stage pulmonary failure, end stage cardiac failure, idiopathic pulmonary fibrosis, polycystic kidneys, and/or short gut syndrome.

The following examples further illustrate various embodiments of the present disclosure, but should not be construed in any way as limiting its scope.

### EXAMPLES

### Example 1

### Defibrotide Treatment in an Autologous Dog Kidney Transplant Model for Ischaemia Reperfusion Injury (IRI)

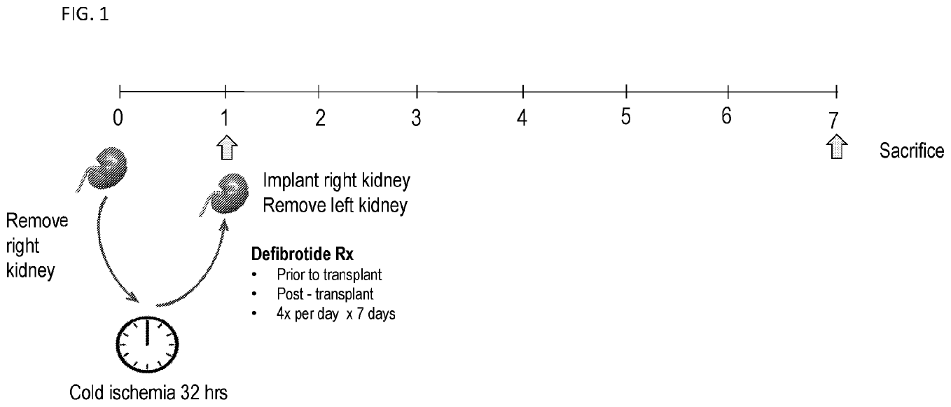

An autologous dog kidney transplant model for IRI was developed (FIG. 1). The right kidney was removed from a dog at day 0 of the study and was exposed to cold ischemia for 32 hours. After exposure to cold ischemia, the right kidney developed IRI. Subsequently, in an autotransplant, the right kidney was implanted, and the left kidney was removed. Defibrotide was administered to the dog prior to transplant and after transplant four times per day for 7 days at doses of 3 mg/kg, 10 mg/kg, or 25 mg/kg. Clinical endpoints were evaluated, including bleeding at the anastomosis site, coagulation, renal function labs, serum creatinine, blood urea nitrogen (BUN), histopathology at sacrifice, and the measurement of biomarkers, including endothelin-1, Kim-1, ICAM-1, E-selectin, t-PA, PAI-1, Thrombomodulin, IL-6, TNF-α, MCP-1, and A2M.

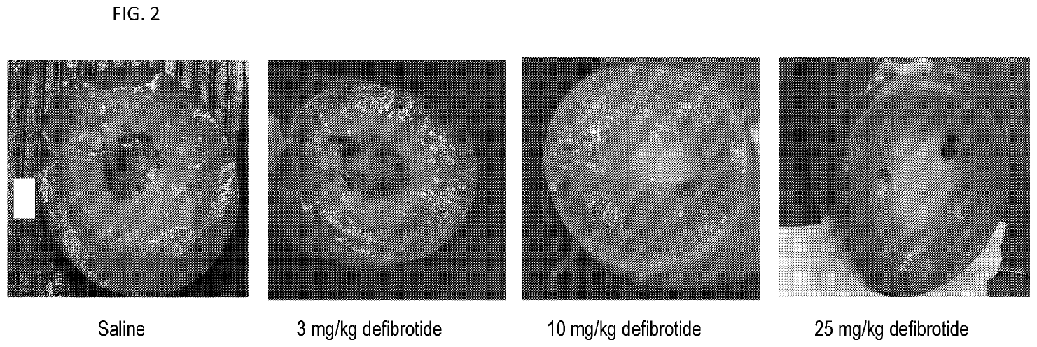

The gross dog kidney morphology was evaluated at the termination of the study. Minimal hemorrhage was observed at higher doses of defibrotide. FIG. 2 shows the dose response effect of defibrotide on the severity of hemorrhage after IRI. After administration of higher doses of defibrotide, the severity of hemorrhage lessened.

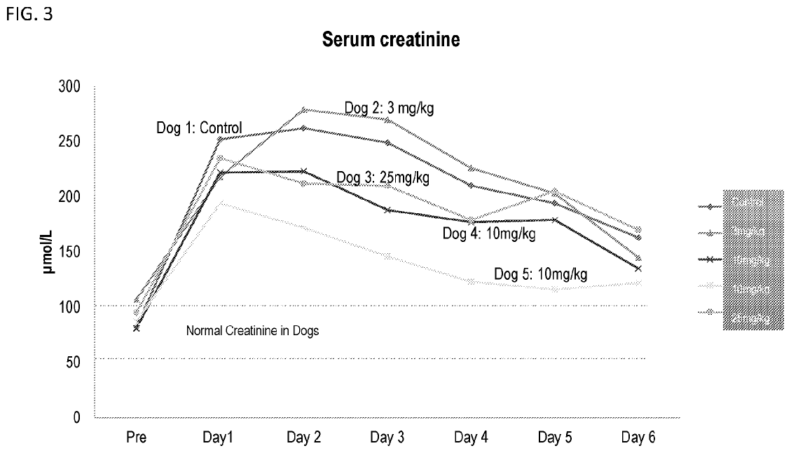

Dogs also exhibited improved renal function with higher doses of defibrotide post IRI. The creatinine level, a marker of renal function, was measured pre-transplant and post-transplant after treatment with saline control, 3 mg/kg, 10 mg/kg, or 25 mg/kg of defibrotide. FIG. 3 shows creatinine levels in the dogs before and after transplant. Each curve represents a different dog. After six days of defibrotide, the creatinine levels of dogs decreased to levels just above normal. Thus, administration of defibrotide improved renal function after transplant.

### Example 2

### Defibrotide to Prevent the Development of Ischaemia Reperfusion Injury in Kidney Transplant Patients

The overall goal of kidney transplant is to achieve the highest possible rates of patient and graft survival and to minimize the incidence of chronic and acute rejection and/or the need for dialysis post-transplant. One limitation of current protocols for organ transplant is damage to the endothelium due to oxidative stress from the time the organ is recovered from the donor, through to transplant and reperfusion in the recipient. The organ tissue is exposed to anaerobic and aerobic respiration during stages of warm and cold ischaemia followed by allo-immune activation in the recipient during reperfusion phase. This ischaemia reperfusion injury (IRI) clinically manifests as delayed or impaired graft function which is associated with a risk for adverse graft outcomes including organ dysfunction and failure. This study assesses the ability of defibrotide to minimize endothelial damage during organ procurement and transplantation. Minimization of endothelial damage can prevent ischaemia reperfusion injury (IRI) leading to improved patient outcomes.

### Clinical Phase 1b Trial:

A multicenter, sequential cohort, dose-escalation study is conducted to assess the safety and efficacy of different schedules of defibrotide for adult patients undergoing DCD kidney transplantation (e.g. transplant from a donor that has experienced cardiac death). About 60 patients are enrolled in the study. The study lasts about 18 months

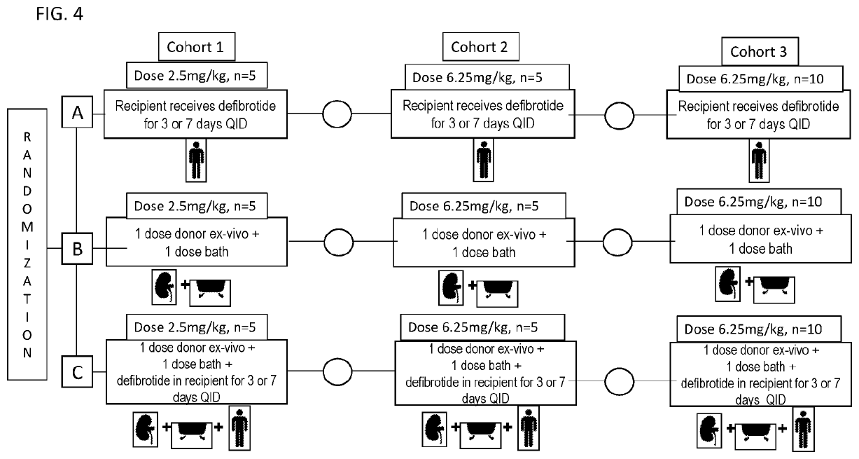

This study contains three arms and three sequential cohorts. Cohort 1 of Arms A, B, and C are described below and summarized in FIG. 4.

a. Arm A: 5 patients will receive defibrotide administered 6-hours post kidney transplantation at doses of 10 mg/kg/day IV and then regularly 4 times per day for 7 days. After patients complete 7 days of treatment, a safety board will review the safety data in accordance with the BOIN rules, make a benefit-risk assessment and provide recommendation to progress to cohort 2

b. Arm B: 5 patients will receive 2.5 mg/kg IV, 1st bolus ex vivo in the donor organ immediately after explantation and 2nd bolus IV delivered into the preservation solution during the initiation of static cold storage. After completion of treatment in 5 patients a safety board will review the safety data in accordance with the BOIN rules, make a benefit-risk assessment and provide recommendation to progress to cohort 2

c. Arm C: 5 patients will receive 2.5 mg/kg IV, 1st bolus ex vivo in the donor organ immediately after explantation and 2nd bolus IV delivered into the preservation solution during the initiation of static cold storage, and then will receive defibrotide administered 6-hours post kidney transplantation at doses of 10 mg/kg/day IV and then regularly 4 times per day for 7 days. After completion of treatment in 5 patients a safety board will review the safety data in accordance with the BOIN rules, make a benefit-risk assessment and provide recommendation to progress to cohort 2

Arms A, B, and C of cohort 2 will receive higher doses of defibrotide (25 mg/kg/day). The administration and dosing of Arms A, B, and C of cohort 2 is described below:

a. Arm A: 5 patients will receive defibrotide administered 6-hours post kidney transplantation at doses of 25 mg/kg/day IV and then regularly 4 times per day for 7 days. After patients complete 7 days of treatment, a safety board will review the safety data in accordance with the BOIN rules, make a benefit-risk assessment and provide recommendation to progress to cohort 3.

b. Arm B: 5 patients will receive 6.25 mg/kg IV, 1st bolus ex vivo in the donor organ immediately after explantation and 2nd bolus IV delivered into the preservation solution during the initiation of static cold storage. After completion of treatment in 5 patients a safety board will review the safety data in accordance with the BOIN rules, make a benefit-risk assessment and provide recommendation to progress to cohort 3.

c. Arm C: 5 patients will receive 6.25 mg/kg IV, 1st bolus ex vivo in the donor organ immediately after explantation and 2nd bolus IV delivered into the preservation solution during the initiation of static cold storage, and then will receive defibrotide administered 6-hours post kidney transplantation at doses of 25 mg/kg/day IV and then regularly 4 times per day for 7 days. After completion of treatment in 5 patients a safety board will review the safety data in accordance with the BOIN rules, make a benefit-risk assessment and provide recommendation to progress to cohort 3

Arms A, B, and C cohort 3 is the expansion cohort which will enroll a further 10 patients following the same treatment schedule as in cohort 2.

All other treatments for kidney transplant recipient will follow standard of care including immunosuppression induction and maintenance and calcineurin therapy will be guided by therapeutic drug monitoring.

The test product used is Defibrotide (Defitelio®) Intravenous solution 200 mg/2.5 mL (80 mg/mL) vial. Study Day 1 is defined as the day of the first defibrotide infusion. In this protocol, the schedule of procedures and assessments will also reference the day relative to the day of transplantation (Day 0). For example, Study Day 1 of this study will also be referred to as transplantation Day −5, whereas the day of transplantation (Day 0) will be referred to as Study Day 6. Transplantation may be delayed for up to 2 days, in which case Transplantation Day 0 will correspond to Study Day 7 (1-day delay) or Study Day 8 (2-day delay).

Inclusion Criteria for subjects enrolled in this study:
1. Patients must be able to understand and sign a written informed consent.
2. Patient is ≥18 years of age at signing of informed consent.
3. Patients must be planned to receive kidney transplant from a DCD deceased donor
4. Has dialysis dependent renal failure initiated at least 60 days prior to transplantation
5. Female patients of childbearing potential and male patients who have female partners of childbearing potential must agree to use a highly effective method of contraception with their partners during exposure to defibrotide and for 1 week after the last dose of defibrotide. Highly effective methods of contraception that may be used by the patient include abstinence (when this is in line with the preferred and usual lifestyle of the patient [periodic abstinence, e.g., calendar, post-ovulation, symptothermal methods, and withdrawal are not acceptable methods]), combined (estrogen- and progestogen-containing) hormonal contraception associated with inhibition of ovulation (i.e., birth control pills, patches, vaginal ring), progestogen-only hormonal contraception associated with inhibition of ovulation (i.e., progestin implant or injection), intrauterine device (IUD), intrauterine hormone-releasing system (IUS), surgical sterilization, and vasectomy (>6 months before study Day 1). Surgically sterile woman and men and post menopausal women (i.e., women with >2 years of amenorrhea) do not need to use contraception.

Exclusion Criteria—Subjects who meet any of the following criteria are excluded from the study:
1. Patient has a planned transplant of kidneys that are implanted en bloc (dual kidney transplant)
2. Patient has planned transplant of dual kidneys transplanted not en bloc (as in the case of dual expanded criteria donor (ECD) donor kidneys)
3. Patient has planned transplant of kidneys from donors <6 years of age
4. Patient is scheduled to receive an HLA or ABO-incompatible kidney (current positive T-cell cross-match)
5. Patient is scheduled to receive a living donor kidney or a kidney from a brain death donor (DBD) donor
6. Patient is scheduled to receive an organ from a donor that meets both DCD and ECD criteria
7. Patient is scheduled to receive an organ with a Kidney Donor Profile Index (KDPI) >80%
8. Patient is scheduled to undergo multi-organ transplantation
9. Patients with historic or current panel reactive antibodies (PRAs) above 25%
10. Previous history of kidney transplantation
11. Previous transplantation with another organ
12. Patients receiving a kidney with cold ischaemic time over 24 h
13. The use of machine perfusion for organ preservation
14. Patient is scheduled for transplantation of a kidney from a donor who is known to have received an investigational therapy (under another IND) for ischemic/reperfusion injury immediately prior to organ recovery
15. Patient is using or plans to use other investigational therapy during this study
16. Patient has participated in another investigational drug study in the last 30 days
17. Anticoagulation planned or anticipated in the peri-operative period
18. Subject plans to use any medication that increases the risk of bleeding, including, but not limited to, systemic heparin, low molecular weight heparin, heparin analogs, alteplase, streptokinase, urokinase, antithrombin III (ATIII), oral anticoagulants including warfarin, and factor Xa inhibitors during the post operative period. Note: Subjects may receive heparin or other anticoagulants for routine central venous line management, and intermittent dialysis or ultrafiltration. Fibrinolytic instillation for central venous line occlusion is also permitted. Heparin use is allowed throughout the study (up to a maximum of 100 U/kg/day).
19. Active bleeding
20. Patients with known prothrombotic disorder
21. Patient, in the opinion of the investigator, may not be able to comply with the study treatment protocol including appropriate supportive care, follow-up, research tests and safety monitoring requirements
22. Patient has a psychiatric illness that would prevent the patient or legal guardian or representative from giving informed consent and/or assent
23. Patient has a serious active disease or co-morbid medical condition, as judged by the investigator, which would interfere with the conduct of this study
24. Patient is pregnant or lactating and does not agree to stop breastfeeding
25. Patient has a known history of hypersensitivity to defibrotide or any of the excipients
26. Previous hypersensitivity to standard of care immunosuppresants e.g. basiliximab,

Campath-1H or antithymocyte globulin (ATG)
27. History of malignancy within the last 5 years (except for successfully excised squamous or basal cell carcinoma of the skin)
28. Presence of clinically significant infections requiring continued therapy
29. Positive screening for active tuberculosis
30. Patient is HCV-positive, HBV (surface antigen)-positive or HIV-positive

Safety: Safety will be assessed through monitoring of AEs, vital signs, physical examinations, and clinical laboratory tests in the recipient. Additional safety assessment of the donor kidney will be performed by visual inspection of the donor kidney following the administration of defibrotide ex-vivo, in the preservation solution and following graft reperfusion. The severity of AEs will be classified by the investigator using the Common Terminology Criteria for Adverse Events (CTCAE), version 5. All subjects will be monitored for AEs for 30 days after the last administration of study drug.

Efficacy will be assessed through monitoring of patient symptoms, physical examinations, laboratory testing, and clinical outcomes. Other assessments include measurement of potential predictive and/or diagnostic biomarkers in blood. Serum biomarkers include CRP, ET-1, ICAM, E-selectin, t-PA, PAI, thrombomodulin, IL-6, TNF-α, MCP-1. Urine biomarkers include cystatin-C, NGAL, MCP-1, IL-18, KIM-1, β2M, urine total protein, and urinary microalbumin. PK blood samples will be collected before and after first defibrotide dose on Day 1 post kidney transplant; and before and after first morning defibrotide dose on Day 7 post kidney transplant at the following timepoints:pre-dose, 1, 2, 3, 4, 5 and 6 hours post dose. Plasma defibrotide concentrations will be measured using a validated bioanalytical method.

The primary objective of the study is to assess the safety and tolerability of different doses and schedules of defibrotide in DCD kidney transplant patients. The primary endpoint of the study is the incidence and severity of adverse events (AE) and changes from baseline of all relevant parameters, including laboratory values. Severity of AEs will be assessed according to CTCAE (version 5)

The secondary objective of the study is to evaluate the preliminary efficacy and pharmacokinetics (PK) of defibrotide for the prevention of Ischaemia Reperfusion Injury (IRI) following DCD kidney transplantation. The secondary endpoints include:

a. Incidence of DGF as defined by the need for acute dialysis within the first 7 days post-transplantation

b. Functional DGF as defined as a failure of the serum creatinine to decrease by at least 10% daily on 3 successive days during the first 7 days post transplant (compared to baseline pre-dialysis creatinine level)

c. Number of dialysis cycles required until dialysis free post transplant

d. Number of days to dialysis free post transplant

e. Total number of dialysis cycles within first 30 days

f. Serum creatinine at baseline pre-dialysis, post transplant day 7, 14, 30, 3-month, 6-month, 12-month

g. Estimated GFR at baseline pre-dialysis, post transplant day 7, 14, 30, 3-month, 6-month, 12-month

h. Incidence of graft loss

i. Patient death

j. Graft and patient survival at 6 and 12-month

k. Incidence and severity of biopsy proven acute rejection

l. Incidence and severity of biopsy proven antibody mediated rejection

m. Number of hospitalizations during follow-up post transplant and cause of hospitalization

n. Plasma blood levels of defibrotide for PK

o. Coagulation parameters: prothrombin time (PT), activated partial thromboplastin time (aPTT), and thrombin time (TT) at baseline, then daily for 7 days post transplant, then at day 14 post transplant

p. Incidence of DGF excluding other non-IRI causes, which include but may not be limited to, the following reasons

q. Treatment of hyperkalaemia or hypervolaemia

r. Hyperacute rejection or other antibody-mediated acute rejection

s. Technical vascular complications involving the allograft: renal arterial and/or venous thrombosis due to vascular injury or technical surgical complications

t. Obstructive uropathy [Radiographically-confirmed]

u. Fulminant recurrence of primary disease (underlying etiology of ESRD) [Biopsy-confirmed], including focal segmental glomerulosclerosis

v. A specific diagnosis of thrombotic microangiopathy (Thrombotic Thrombocytopenic Purpura or Hemolytic—Uremic Syndrome). [Biopsy-confirmed]

The exploratory objective of the study is to evaluate the presence of biomarkers before, after, and during treatment with defibrotide. The exploratory endpoints include urine biomarkers, such as Cystatin-C, NGAL, MCP-1, IL-18, KIM-1, B2M, urine total protein, urinary microalbumin, and serum biomarkers, such as CRP, ET-1, ICAM , E-Selectin, t-PA, PAI, Thrombomodulin, IL-6, TNF-α, and MCP-1.

Sequential dose escalation will allow the assessment of safety and efficacy for different doses and schedules of defibrotide in the prevention of IRI in deceased donor adult kidney transplantation.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

This application incorporates by reference the following publications, patents and applications in their entireties for all purposes: U.S. application Ser. No. 16/105,319 filed Aug. 3, 2018; PCT/US2019/064901 filed December 6, 2019 and PCT/US2021/019964 filed Feb. 26, 2021, and PCT/US2019/027210 filed on Apr. 12, 2019, and Ser. No. 16/398,978, filed on Apr. 30, 2019; as well as U.S. Pat. Nos. 3,770,720, 3,829,567, 3,899,481, 4,649,134, 4,693,995, 4,938,873, 4,985,552, 5,081,109, 5,116,617, 5,223,609, 5,646,127, 5,646,268, and 6,046,172.

### REFERENCES

1. Scientific Registry of Transplant Recipients. https://www.srtr.org/2.
2. Hart A, Smith Jm, Skeans M A, et al. OPTN/SRTR 2016 Annual Data Report: Kidney. Am J Transplant. 2018; Volume 18, IssueS1; 18-113
3. Rao P S and Ojo A. The Alphabet Soup of Kidney Transplantation: SCD, DCD, ECD—Fundamentals for the Practicing Nephrologist. Clin J Am Soc Nephrol. 2009; 4: 1827-1831
4. Irish W D, Ilsley J N, Schnitzler M A, Feng S, Brennan D C. A risk prediction model for delayed graft function in the current era of deceased donor renal transplantation. Am J Transplant. 2010; 10: 2279-b 2286
5. Cavaille-Coll M, Bala S, Velidedeoglu E, et al. Summary of FDA Workshop on Ischemia Reperfusion Injury in Kidney Transplantation. Am J Transplant. 2013; 13: 1134-1148
6. Stewart D, Kucheryavaya A, Klassen D. Understanding the Drivers Behind an Early Post-KAS Rise in Delayed Graft Function (DGF) Rates. Am J Transplant. 2016; 16 (suppl 3)
7. Carden D L and Granger D N. Pathophysiology of ischaemia-reperfusion injury. J Pathol. 2000; 190: 255-266
8. Jang H R and Rabb H. The innate immune response in ischemic acute kidney injury. Clin Immunol. 2009; 130: 41-50
9. Sharfuddin A A and Molitoris B A. Pathophysiology of ischemic acute kidney injury. Nat Rev Nephrol. 2011; 7 (4): 189-200
10. Cardinal H, Dieudé M and Hebert M-J. Endothelial Dysfunction in Kidney Transplantation. Front. Immunol. 2018; 9: 1130
11. Zens T, Leverson G, Redfield R, et al. The Impact of Kidney Donor Profile Index (KDPI) on Rates of Delayed Graft Function (DGF). Am J Transplant. 2016; 16 (suppl 3)
12. Boom H, Mallat M J, de Fijter JW, Zwinderman A H, Paul L C. Delayed graft function influences renal function, but not survival. Kidney Int. 2000; 58: 859-866
13. Miglinas M, Supranaviciene L, Mateikaite K, Skebas K, Kubiliene A. Delayed graft function: risk factors and the effects of early function and graft survival. Transplant Proc. 2013; 45: 1363-1367
14. Lim W H, McDonald S P, Russ G R et al. Association Between Delayed Graft Function and Graft Loss in Donation After Cardiac Death Kidney Transplants—A Paired Kidney Registry Analysis. Transplantation. 2017; 101: 1139-1143
15. Global Observatory on Donation and Transplant, Organ Donation and Transplantation Activities, 2015 Report. P 22, Sep. 2017. http://www.transplant-observatory.org/organ-donation-transplantation-activities-2015-report/
16. NHS Blood and Transplant, Organ Donation and Transplantation Activity Report 2016/17, p 46
17. Geographic Variation in Cold Ischemia Time: Kidney Versus Liver Transplantation in the United States, 2003 to 2011, Transplant Direct, August 2015
18. Hart et al, OPTN/SRTR 2014 Annual Data Report, Kidney, American Journal of Transplantation 2016, p 13
19. Hart et al, OPTN/SRTR 2014 Annual Data Report, Kidney, American Journal of Transplantation 2016, p 14
20. Bonomini V, Vangelista A, Stefoni S, et al. Use of Defibrotide in Renal Transplantation in Man. Haemostasis. 1986; 16 supple. 1; 48-50
21. Frasca G M, Vangelista A, Raimondi C, Bonomini V. Prevention of Vascular Graft Lesions in Renal Transplant Recipients with a New Antithrombotic Agent (Defibrotide): a Controlled Study. Life Support Systems. 1986; 4: 231-237
22. Bonomini, V. Frasca, G. M., Raimondi, C., Liviano D' arcangelo, Vangelista, A. Effect of a New Antithrombotic Agent (Defibrotide) in Acute Renal Failure Due to Thrombotic Microangiopathy. 1985. Nephron, 40: 195-200.
23. Jing et al. Organ preservation: from the past to the future. Acta Pharmacologica Sinica (2018) 39: 845-857.
24. Petrenko et al. Organ Preservation into the 2020s: The Era of Dynamic Intervention. Transfus Med Hemother 2019; 46:151-172.
25. Guibert et al. Organ Preservation: Current Concepts and New Strategies for the next Decade.Transfus Med Hemother 2011; 38:125-142.

1. A method of preventing and/or lessening the effects of and/or treating organ injury and/or organ rejection in a patient comprising administering a therapeutically effective amount of defibrotide.

2. The method of claim 1, wherein the patient is receiving or about to receive or has received transplant of a donor organ or donor tissue.

3. The method of claim 2, wherein the donor organ is selected from the group consisting of a heart, heart valve, stomach, testis, liver, lungs, kidneys, pancreas, uterus, intestine, bladder, composite tissue, xenograft, and thymus, and/or wherein the donor tissue is selected from the group consisting of bones, bone marrow, tendons, corneae, skin, valves, nerves, and veins.

4. The method of claim 2, wherein defibrotide is administered to the patient, to the donor organ intravenously via an artery, or to a preservation bath that is used to store the organ for transfer.

5. (canceled)

6. (canceled)

7. The method claim 1, wherein the defibrotide is administered before the development of organ injury and/or organ rejection.

8. (canceled)

9. The method of claim 1, wherein defibrotide is administered to the patient once or four times per day.

10. The method of claim 1, wherein defibrotide is administered to the patient in multiple doses per day.

11. The method of claim 1, wherein defibrotide is administered in two to ten doses per day.

12. (canceled)

13. The method of claim 1, wherein an initial dose of defibrotide is administered to the patient about 6 hours after transplant of a donor organ.

14. The method of claim 1, wherein the defibrotide is administered at a dose between 1 mg/kg and 10 mg/kg.

15. The method of claim 14, wherein the defibrotide is administered at a dose of 2.5 mg/kg or 6.25 mg/kg.

16. (canceled)

17. The method of claim 1, wherein the defibrotide is administered intravenously or subcutaneously.

18. (canceled)

19. The method of claim 1, wherein defibrotide is administered intravenously, every six hours at a dose of 2.5 mg/kg or 6.25 mg/kg.

20. (canceled)

21. The method of claim 1, wherein the defibrotide is administered to the donor organ intravenously via an artery at a dose of 2.5 mg/kg or 6.25 mg/kg, or to a preservation bath that is used to store the organ for transfer at a dose of 2.5 mg/kg or 6.25 mg/kg.

22. (canceled)

23. The method of claim 1, wherein the defibrotide is a high concentration defibrotide formulation.

24. The method of claim 23, wherein the high concentration defibrotide formulation is formulated for subcutaneous delivery or intravenous delivery.

25. The method of claim 23, wherein the high concentration defibrotide formulation comprises about 160-200 mg/mL of defibrotide and about 20 mM glycylglycine, and is formulated for subcutaneous delivery to a patient.

26. The method of claim 23, wherein the high concentration defibrotide formulation further comprises between about 10 mM to about 34 mM sodium citrate.

27. The method of claim 23, wherein the high concentration defibrotide formulation comprises about 160-200 mg/mL of defibrotide, about 20 mM glycylglycine, and about 10-25 mM sodium citrate, wherein the formulation is formulated for subcutaneous or intravenous delivery to a patient.

28. The method of claim 1, wherein the organ injury is selected from the group consisting of interstitial fibrosis, tubular atrophy, glomerulosclerosis, acute kidney injury, diffuse alveolar damage, nonalcoholic steatohepatitis, fibrosis, cardiac allograph vasculopathy, and ischemia reperfusion injury (IRI).
